(19) United States
(12) Patent Application Publication
(10) Pub. No.: US 2006/0148866 A1
(43) Pub. Date: Jul. 6, 2006

(54) Novel process for preparing pramipexole and its optical isomeric mixture by reduction with sodium triacetoxyborohydride

(75) Inventors: Guangxin Xia, Shanghai (CN); Yifeng Nian, Shanghai (CN); Tiema Yan, Shanghai (CN); Jin Suo, Shanghai (CN); Michael Brand, RaAnana (IL); Oded Arad, Rechovot (IL)

Correspondence Address: Martin Moynihan;PRTSI, Inc., P.O. Box 16446, Arlington, VA, 22215, US

(73) Assignee: Chemagis Ltd., Bnei Brak (IL)

(21) Appl. No.: 11/299,752

(22) Filed: Dec. 13, 2005

Related U.S. Application Data
(60) Provisional application No. 60/640,012, filed on Dec. 30, 2004.

(51) Int. Cl. C07D 277/82; A61K 31/428

(52) U.S. Cl. 514/367; 548/161

(57) ABSTRACT

A novel process is provided for producing pramipexole base or its optical isomeric mixture as defined hereinabove i.e. (R,S)-2-amino-6-propyl-4,5,6,7-tetrahydrobenzothiazole avoiding the use of borane tetrahydrofuran complex and using a more convenient reducing agent like sodium triacetoxyborohydride instead. The provided process comprises reacting the starting material (S)-2,6-diamino-4,5,6,7-tetrahydrobenzothiazole or its optical isomeric mixture as defined hereinabove i.e. (R,S)-2,6-diamino-4,5,6,7-tetrahydrobenzothiazole with propionaldehyde in an organic solvent to obtain the respective enamine, which is subsequently reduced in situ, optionally without isolation, to obtain pramipexole or its optical isomeric mixture as defined hereinabove i.e. (R,S)-2-amino-6-propyl-4,5,6,7-tetrahydrobenzothiazole, and the acid addition salts thereof. The present invention also provides a process for purifying pramipexole dihydrochloride or the dihydrochloride salt of its optical isomeric mixture as defined hereinabove i.e. (R,S)-2-amino-6-propyl-4,5,6,7-tetrahydrobenzothiazole dihydrochloride by re-crystallization from a suitable solvent.

### RELATED APPLICATIONS

The present application claims the benefit of U.S. Provisional Patent Application No. 60/640,012, filed on Dec. 30, 2004, which is incorporated herein by reference in its entirety.

### FIELD OF THE INVENTION

The present invention provides a novel process for preparing pramipexole and its optical isomeric mixture, using the mild reducing agent sodium triacetoxyborohydide, thus avoiding the use of borane tetrahydrofuran complex (BTHF).

### BACKGROUND OF THE INVENTION

(S)-2-amino-6-propylamino-4,5,6,7-tetrahydrobenzothiazole dihydrochloride, more commonly known as pramipexole dihydrochloride, is a synthetic aminobenzothiazole derivative having the structural formula 1, which is marketed under the trade name Mirapex®.

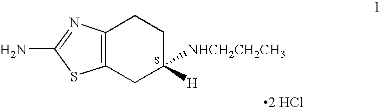

### Pramipexole Dihydrochloride

The drug is a dopamine agonist used for treating Parkinson's disease by stimulating the dopamine receptors in the brain.

Various synthetic routes for preparing pramipexole, its salts thereof and the intermediates thereof were previously described in European Patent Nos. 186087 and 207696; U.S. Pat. Nos. 6,727,367 and 6,770,761; and PCT Publications Nos. WO 2004/026850, WO 2004/041797 and WO 2005/014562. An additional synthetic route was disclosed by C. S Schneider and J. Mierau in J. Med. Chem., 1987, 30, 494-498. According to this route, pramipexole may be prepared by reacting (S)-2-amino-6-propionamido-4,5,6,7-tetrahydrobenzothiazole, a compound of formula 2, with borane tetrahydrofuran complex (BTHF) in the presence of anhydrous THF to yield (S)-2-amino-6-propylamino-4,5,6,7-tetrahydrobenzothiazole base, a compound of formula 3. The isolated base is consequently converted into the dihydrochloride salt, which is re-crystallized from methanol in an overall yield of 50%. This process is illustrated in Scheme 1:

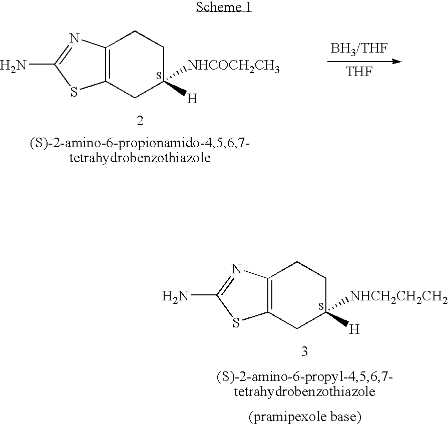

This synthetic route involves using the reducing agent BTHF, which is supplied as a 1.0 M or 1.5 M solution in THF. The reagent is thermally unstable and must be stored in the cold (below 5° C.). Furthermore, BTHF is susceptible to hydrolysis, readily reacting with water to form hydrogen and boric acid and readily reacting with atmospheric moisture upon exposure to air, resulting in a decrease in assay. At elevated temperatures of above 50° C. and in the absence of a substrate BTHF decomposes by cleavage of the ether ring to evolve the diborane gas, which is extremely toxic. In addition, tetrahydrofuran can form potentially explosive peroxides upon long standing in the air.

All the above restrictions and warnings make the use of BTHF complicated, expensive (due to high freight and storage costs), inconvenient and environmentally harmful and it appears clear that this process cannot be advantageously used for large-scale production.

Due to this problematic implementation of BTHF in large-scale preparations, there is an unmet need in the art for a more convenient and economically feasible process that will use an alternative safer reducing reagent, which will be more stable for synthetic applications. Thus, the object of the present invention is to provide an improved process for preparing pramipexole, which avoids the use of borane tetrahydrofuran complex.

In the U.S. patent application, entitled “An improved process for the reduction of (S)-2-amino-6-propionamido-4,5,6,7-tetrahydrobenzothiazole”, by the present inventors, which claims priority from U.S. Provisional Patent Application No. 60/614,422 (filed on Sep. 29, 2004), which is incorporated by reference as if fully set forth herein, an improved process is disclosed for the reduction of (S)-2-amino-6-propionamido-4,5,6,7-tetrahydrobenzothiazole, an intermediate useful in the preparation of (S)-2-amino-6-propylamino-4,5,6,7-tetrahydrobenzothiazole, more commonly known as pramipexole.

The present application affords an alternative synthetic route for preparing pramipexole base and its optical isomeric mixture, which is more advantageous over the existing processes for preparing pramipexole base.

### SUMMARY OF THE INVENTION

In one embodiment, the present invention provides a novel process for preparing pramipexole base, the process comprising reacting the starting material (S)-2,6-diamino-4,5,6,7-tetra-hydrobenzothiazole with propionaldehyde in an organic solvent to obtain an enamine (compound 5 in Scheme 2 below), which is reduced in situ, optionally without prior isolation, using the reducing agent sodium triacetoxyborohydride [NaB(O₂CCH₃)H], thus the usage of borane tetrahydrofuran complex is avoided.

According to the present invention, the process using the reducing agent sodium triacetoxyborohydride is applicable also for reacting (R,S)-2,6-diamino-4,5,6,7-tetrahydrobenzothiazole, which is hereinunder referred to as the optical isomeric mixture of the starting material (S)-2,6-diamino-4,5,6,7-tetrahydrobenzo-thiazole for preparing (R,S)-2-amino-6-propyl-4,5,6,7-tetrahydrobenzothiazole, which is hereinunder referred to as the optical isomeric mixture of pramipexole base.

In another embodiment of the present invention, there is provided a procedure of isolating the crude pramipexole base or its optical isomeric mixture as defined hereinabove, the procedure comprising:

evaporating the reaction mixture to dryness, optionally under reduced pressure;

adding a basic aqueous solution and an organic solvent to form a two-phase system, extracting and separating the phases, and washing the organic phase;

evaporating the solvent to dryness to obtain an oily residue;

adding an organic solvent and suspending the mixture optionally at elevated temperature; and

precipitating the crude product, collecting it by filtration, washing and drying.

In yet another embodiment of the present invention, once the reaction is complete the product, which is either crude pramipexole base or its optical isomeric mixture as defined hereinabove, may be converted into an acid addition salt such as the dihydrochloride salt and isolated in solid state by methods described herein.

In yet another embodiment of the present invention pramipexole dihydrochloride or the dihydrochloride salt of the optical isomeric mixture as defined hereinabove may be purified by re-crystallization process from a suitable solvent.

### DESCRIPTION OF THE PREFERRED EMBODIMENTS

The present invention provides a novel process for preparing pramipexole base or its optical isomeric mixture i.e. (R,S)-2-amino-6-propyl-4,5,6,7-tetrahydrobenzo-thiazole, and the addition salts thereof, avoiding the use of borane tetrahydrofuran complex (BTHF) and using an alternative, more convenient reducing agent instead. The inventors of the present invention have studied the use of other reducing agents instead of borane tetrahydrofuran complex for preparing pramipexole. Unsatisfactory results were obtained while using sodium borohydride (NaBH₄), due to relatively low conversion, because the crude product contained about 20% of the starting material (R,S)-2,6-diamino-4,5,6,7 tetrahydrobenzothiazole (see Example 4).

Another reagent that has been tested for obtaining pramipexole base or its optical isomeric mixture as defined hereinabove was lithium aluminium hydride (LiAlH₄). This reagent is not preferred for industrial application because the mixture of the reagent (LiAlH₄) and the solvent (THF) is highly flammable. In addition a 14-fold excess of the reagent was needed in order to achieve high conversion. Furthermore, it has been found that it is very difficult to purify the crude product without using column chromatography, as can be seen in the Examples section that follows (see Example 5).

It has been surprisingly discovered by the inventors of the present invention that a preferred reducing agent is solid sodium triacetoxyborohydride [NaB(O₂CCH₃)H]. Using solid sodium triacetoxyborohydride is advantageous not only because it is non-toxic and safer for use in comparison to the borane THF complex, but also because the handling of a solid is much simpler in comparison to a hazardous solution, in addition to being less expensive due to the lowered freight and storage costs.

In a preferred embodiment of the present invention, the reducing agent sodium triacetoxyborohydride is used for preparing pramipexole base ((S)-2-amino-6-propyl-4,5,6,7-tetrahydrobenzothiazole).

According to the present invention, the process comprises reacting the starting material (S)-2,6-diamino-4,5,6,7 tetrahydrobenzothiazole in an organic solvent to obtain an enamine (compound 5 in Scheme 2 below), which is reduced in situ, optionally without prior isolation, thus the usage of borane tetrahydrofuran complex is avoided. The process is described in Scheme 2 below.

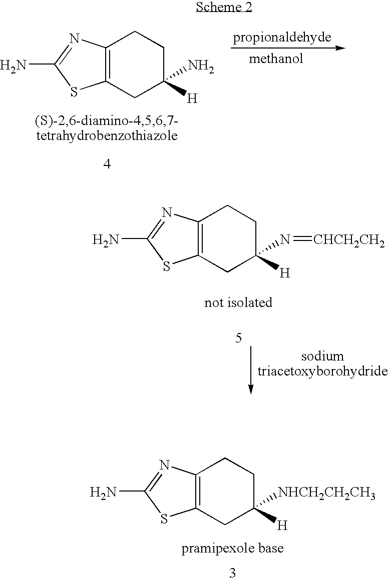

According to the present invention, the process using the reducing agent sodium triacetoxyborohydride is applicable also for reacting (R,S)-2,6-diamino-4,5,6,7-tetrahydrobenzothiazole, which is hereinunder referred to as the optical isomeric mixture of the starting material (R,S)-2,6-diamino-4,5,6,7-tetrahydrobenzothiazole for preparing the optical isomeric mixture of pramipexole base, as defined hereinabove.

Thus, the present invention provides a process for preparing pramipexole base or its optical isomeric mixture as defined hereinabove, comprising:

reacting (S) or (R,S)-2,6-diamino-4,5,6,7-tetrahydrobenzothiazole with propionaldehyde in a suitable organic solvent to obtain the respective enamine in situ;

reacting the enamine thus formed in situ, optionally without isolation, with sodium triacetoxyborohydride in the presence of a suitable organic solvent to yield pramipexole base or its optical isomeric mixture as defined hereinabove;

quenching the reaction mixture by adding an aqueous acidic solution; and

isolating the pure product as a free base or as an acid addition salt thereof.

According to one embodiment of the present invention, the process is carried out in a suitable organic solvent selected from the group consisting of tetrahydrofuran, acetic acid, acetonitrile, ethyl acetate, methanol, ethanol, 1-propanol, 2-propanol, and mixtures thereof. The presently most preferred solvent is methanol.

According to another embodiment of the present invention the process is conducted at a temperature range of between 0° C. and ambient temperature, preferably at 5° C., for a short reaction time, preferably of about an hour.

According to yet another embodiment of the present invention, the reaction is quenched by addition of an aqueous acidic solution preferably a solution of 10% HCl.

According to another preferred embodiment of the present invention the isolation procedure of obtaining crude pramipexole base or its optical isomeric mixture as defined hereinabove comprising:

evaporating the reaction mixture to dryness, optionally under reduced pressure;

adding a basic aqueous solution and an organic solvent to form a two-phase system, extracting and separating the phases, and washing the organic phase;

evaporating the solvent to dryness to obtain an oily residue;

adding an organic solvent and suspending the mixture optionally at elevated temperature; and

precipitating the crude product, collecting it by filtration, washing and drying.

According to another embodiment of the present invention, evaporating the reaction mixture to dryness is carried out at a temperature of no more than 50° C.

According to yet another embodiment of the present invention, the basic aqueous solution is selected from the group consisting of aqueous solutions of sodium hydroxide, potassium hydroxide, sodium carbonate, potassium carbonate, sodium bicarbonate, and potassium bicarbonate. The presently most preferred basic aqueous solution is an aqueous solution of sodium hydroxide.

According to yet another embodiment of the present invention, the concentration of the said aqueous solution of sodium hydroxide is at least 10%, preferably of about 45%.

According to yet another embodiment of the present invention, the organic solvent used in the isolation procedure is selected from the group consisting of methyl acetate, ethyl acetate, propyl acetate, isopropyl acetate, butyl acetate, isobutyl acetate, toluene, dichloromethane, chloroform, and mixtures thereof. The presently most preferred solvent is ethyl acetate.

According to yet another embodiment of the present invention, the isolated product may be dried by using conventionally known methods to give pure pramipexole base or its optical isomeric mixture as defined hereinabove. The drying procedure may be carried out by increasing the temperature or by reducing the pressure or a combination of both. Non limiting examples of drying technologies or equipment usable in context of the present invention include rotary evaporators, vacuum ovens, tray ovens, rotary ovens, and fluidized bed dryers.

In yet another embodiment of the present invention, pramipexole base or its optical isomeric mixture as defined hereinabove may be converted into an acid addition salt without isolation of the free base, i.e. in the same reaction vessel. Alternatively, pramipexole base or its optical isomeric mixture as defined hereinabove may be converted into an acid addition salt after being isolated from the reaction mixture. Preferably, these salts are pharmaceutically acceptable salts. The conversion of pramipexole base or its optical isomeric mixture as defined hereinabove may be accomplished by treatment with at least a stoichiometric amount of an appropriate acid.

According to the present invention, the appropriate acid includes, but is not limited to, inorganic acids such as hydrochloric acid and the like, and organic acids such as tartaric acid and the like.

According to yet another embodiment of the present invention, there is provided a procedure for converting pramipexole base to pramipexole dihydrochloride or its optical isomeric mixture as defined hereinabove i.e. (R,S)-2-amino-6-propyl-4,5,6,7-tetrahydrobenzothiazole, to its dihydrochloride salt, the procedure comprising:

dissolving pramipexole base or its optical isomeric mixture as defined hereinabove i.e. (R,S)-2-amino-6-propyl-4,5,6,7-tetrahydrobenzothiazole in a suitable solvent and filtering the solution to obtain a filtrate;

adding a solution of an inorganic acid in an organic solvent and mixing;

cooling to a reduced temperature and mixing to obtain a precipitate; and

collecting the precipitate by filtration, washing and drying.

According to yet another embodiment of the present invention, the suitable solvent used for dissolving pramipexole base or its optical isomeric mixture as defined hereinabove is selected from the group consisting of methanol, ethanol, 1-propanol, 2-propanol, water, and mixtures thereof. The presently most preferred solvent is ethanol.

According to yet another embodiment of the present invention, the preferred solution of an inorganic acid in an organic solvent is a solution of at least 10% HCl in 2-propanol and preferably about 14.6% HCl in 2-propanol.

In yet another embodiment of the present invention, the pramipexole dihydrochloride or the dihydrochloride salt of its optical isomeric mixture as defined hereinabove may be re-crystallized by any conventional re-crystallization method known in the art.

In yet another embodiment of the present invention, pramipexole dihydrochloride or the dihydrochloride salt of its optical isomeric mixture as defined hereinabove may be purified by re-crystallization process from a suitable solvent, the process comprising:

converting pramipexole dihydrochloride or the dihydrochloride salt of its optical isomeric mixture as defined hereinabove i.e. (R,S)-2-amino-6-propyl-4,5,6,7-tetrahydrobenzothiazole dihydrochloride to the corresponding free base by treatment with at least a stoichiometric equivalent of a suitable organic or inorganic base;

converting the pure free base product again to the corresponding pramipexole dihydrochloride or (R,S)-2-amino-6-propyl-4,5,6,7-tetrahydrobenzothiazole dihydrochloride; and

isolating the purified pramipexole dihydrochloride or the dihydrochloride salt of its optical isomer as defiened hereinabove i.e. (R,S)-2-amino-6-propyl-4,5,6,7-tetrahydrobenzothiazole dihydrochloride.

In yet another embodiment of the present invention, the processes described herein for obtaining pramipexole base or its optical isomeric mixture as defined hereinabove may be conveniently and inexpensively scaled-up.

Accordance to the present invention, the following are the advantages of the process for preparing pramipexole base or its optical isomeric mixture as defined hereinabove and the acid addition salts thereof provided herein:

1) Using solid sodium triacetoxyborohydride is advantageous because it is non-toxic and safer for use in comparison to the borane THF complex.

2) Sodium triacetoxyborohydride is a solid material; therefore its handling is much simpler in comparison to a hazardous solution, in addition to being less expensive due to the lowered freight and storage costs.

3) Only about 1.4-fold excess of the reagent sodium triacetoxyborohydride relative to the starting material is used in comparison to a 14-fold excess used in the case of LiAlH₄ (Example 5).

4) The reaction yield while using the reagent sodium triacetoxyborohydride is much higher than the yield reported in the reduction with NaBH₄, which is 42% as reported in European Patent No. 186087 (example 7).

Although the following examples illustrate the practice of the present invention in some of its embodiments, the examples should not be construed as limiting the scope of the invention. Other embodiments will be apparent to one skilled in the art from consideration of the specification and examples. It is intended that the specification, including the examples, is considered exemplary only, with the scope and spirit of the invention being indicated by the claims that follow.

### EXAMPLES

### Example 1

A reaction vessel equipped with a magnetic stirrer and a thermometer was charged with (S)-2,6-diamino-4,5,6,7-tetrahydrobenzothiazole (5.0 g, 0.0296 mole) and methanol (100 ml) and the solution was cooled to 5° C. under constant stirring. A solution of propionaldehyde (2.7 ml) in methanol (10 ml) was added while maintaining the inner temperature at 5° C. The reaction mixture was stirred at this temperature for 15 minutes. Sodium triacetoxyborohydride (8.75 g, 0.0413 mole) was added in 4-5 portions while maintaining the temperature at 5° C. Stirring was continued at 5° C. for 5 minutes and the mixture was allowed to warm to 25° C. during about 30 minutes.

A mixture of water (100 ml) and 32% HCl solution (30 ml) was added to the reaction mixture to afford a suspension. The reaction mixture was evaporated to dryness under reduced pressure keeping the bath temperature at less then 50° C. Water (20 ml) and ethyl acetate (60 ml) were added to afford a two-phase system, and 45% aqueous sodium hydroxide solution (4.5 ml) was added. The layers were separated and the upper organic layer was washed with water (2×10 ml).

The ethyl acetate solution was evaporated to dryness keeping the bath temperature at less than 50° C. to afford an oily residue. Ethyl acetate (7 ml) was added to the oily residue and the suspension thus formed was stirred at 50° C. for 15 minutes. The mixture was allowed to cool to 25° C. and stirred at this temperature for 1 hour. Then, the mixture was cooled down to 5° C. and stirred at this temperature for 1 hour.

The precipitate thus formed was filtered, washed with cold ethyl acetate and dried at 60° C. to yield 4.3 g (69%) of pramipexole base.

### Example 2

A reaction vessel equipped with a magnetic stirrer and a thermometer was charged with (R,S)-2,6-diamino-4,5,6,7-tetrahydrobenzothiazole (5.0 g, 0.0296 mole) and methanol (100 ml) and the solution was cooled to 5° C. under constant stirring. A solution of propionaldehyde (2.7 ml) in methanol (10 ml) was added while maintaining the inner temperature at 5° C. The reaction mixture was stirred at this temperature for 15 minutes. Sodium triacetoxyborohydride (8.75 g, 0.0413 mole) was added in 4-5 portions while maintaining the temperature at 5° C. Stirring was continued at 5° C. for 5 minutes and the mixture was allowed to warm to 25° C. during about 30 minutes.

A mixture of water (100 ml) and 32% HCl solution (30 ml) was added to the reaction mixture to afford a suspension. The reaction mixture was evaporated to dryness under reduced pressure keeping the bath temperature at less then 50° C. Water (20 ml) and ethyl acetate (60 ml) were added to afford a two-phase system, and 45% aqueous sodium hydroxide solution (4.5 ml) was added. The layers were separated and the upper organic layer was washed with water (2×10 ml).

The ethyl acetate solution was evaporated to dryness keeping the bath temperature at less than 50° C. to afford an oily residue. Ethyl acetate (7 ml) was added to the oily residue and the suspension thus formed was stirred at 50° C. for 15 minutes. The mixture was allowed to cool to 25° C. and stirred at this temperature for 1 hour. Then, the mixture was cooled down to 5° C. and stirred at this temperature for 1 hour.

The precipitate thus formed was filtered, washed with cold ethyl acetate and dried at 60° C. to yield 4.3 g (69%) of pramipexole base.

### Example 3

A reaction vessel equipped with a magnetic stirrer was charged with pramipexole base (2.53 g) and absolute ethanol (20 ml). The mixture was stirred at room temperature to afford a clear solution. The solution was filtered and the filtrate was transferred to another reaction vessel. A solution of about 14.6% HCl in 2-propanol (7.8 ml) was added in portions and the resulting mixture was stirred for 1 hour. The mixture was cooled to about 5° C. and stirred for additional 1 hour. The precipitate was filtered, washed with cold ethanol and dried at 60° C. under vacuum to yield 3.0 g (89%) of pramipexole dihydrochloride.

### Example 4

A reaction vessel equipped with a magnetic stirrer and a thermometer was charged with (R,S)-2,6-diamino-4,5,6,7-tetrahydrobenzothiazole (10.0 g, 0.0592 mole) and methanol (200 ml) and the solution was stirred and cooled to about −15° C. A solution of propionaldehyde (5.4 ml) in methanol (20 ml) was added keeping the inner temperature at about −15° C. The reaction mixture was stirred at this temperature for 60 minutes. Sodium borohydride (3.1 g, 0.082 mole) was added in 4-5 portions during a period of about 30 minutes while maintaining the temperature at −15° C. Stirring was continued at −15° C. for 5 minutes and the mixture was allowed to warm to about 25° C. during about 30 minutes and stirred at this temperature for 1 hour.

A sample was withdrawn and injected to HPLC. The conversion of (R,S)-2-amino-6-propyl-4,5,6,7-tetrahydrobenzothiazole was 55% and 20% of (R,S)-2,6-diamino-4,5,6,7-tetrahydrobenzothiazole was also detected.

### Example 5

A reaction vessel equipped with a magnetic stirrer and a thermometer was charged with THF (225 ml) and with (R,S)-6-acetylamino-2-amino-4,5,6,7-tetrahydrobenzothiazole (4.5 g, 0.02 mole).

LiAlH₄ (1.48 g, 0.04 mole) was added and the mixture was heated to 45° C. 6 additional portions of LiAlH₄ (6×1.48 g, 0.24 mole) were added at intervals of 3 hours between each addition. (The total LiAlH₄ quantity added was 10.36 g, 0.28 mole). Before adding each portion of LiAlH₄, the reaction mixture was cooled down to 20° C. and heated again to 45° C. for about 15 minutes after completing the addition. About 24 hours after reaction was started, the reaction mixture was cooled down to about 0° C. and a mixture of 80:20 (v/v) THF:water (100 ml) was added. The suspension was concentrated by evaporation. The residue thus formed was mixed with methanol (200 ml) and refluxed for 1 hour. The solid was filtered off and the filtrate was concentrated by evaporation.

The residue was purified by column chromatography on silica gel (eluent:methylene chloride/methanol=80/20). The corresponding fraction was concentrated by evaporation. (R,S)-2-amino-6-propyl-4,5,6,7-tetrahydrobenzothiazole dihydrochloride was crystallized out while a solution of hydrochloric acid in 2-propanol was added to yield 3 g (60% of the theoretical quantity), m.p. 260° C.

What is claimed is:

1. A process for preparing tetrahydrobenzothiazole compounds selected from the group consisting of pramipexole base or its optical isomeric mixture as defined hereinabove i.e. (R,S)-2-amino-6-propyl-4,5,6,7-tetrahydrobenzothiazole, avoiding using borane tetrahydrofuran complex, the process comprising:
reacting (S)-2,6-diamino-4,5,6,7-tetrahydrobenzothiazole or its optical isomeric mixture as defined hereinabove i.e. (R,S)-2,6-diamino-4,5,6,7-tetrahydrobenzothiazole with propionaldehyde in a suitable organic solvent to obtain the respective enamine in situ;
reacting the enamine thus formed in situ, optionally without isolation, with sodium triacetoxyborohydride in the presence of a suitable organic solvent to yield pramipexole base or its optical isomeric mixture as defined hereinabove;
quenching the reaction mixture by adding an aqueous acidic solution; and
isolating the pure product as a free base or as an acid addition salt thereof.

2. The process according to claim 1, wherein the suitable organic solvent is selected from the group consisting of tetrahydrofuran, acetic acid, acetonitrile, ethyl acetate, methanol, ethanol, 1-propanol, 2-propanol, and mixtures thereof.

3. The process according to claim 2, wherein the suitable organic solvent is methanol.

4. The process according to claim 1, wherein said reacting is conducted at a temperature ranging between 0° C. and ambient temperature, preferably at 5° C., for a short time, preferably of about one hour.

5. The process according to claim 1, wherein said aqueous acidic solution is a solution of about 10% HCl.

6. The process according to claim 1, wherein the said isolating procedure of crude pramipexole base or its optical isomeric mixture as defined hereinabove i.e. (R,S)-2-amino-6-propyl-4,5,6,7-tetrahydrobenzothiazole comprising:
evaporating the reaction mixture to dryness, optionally under reduced pressure;
adding a basic aqueous solution and an organic solvent to form a two-phase system, extracting and separating the phases, and washing the organic phase;
evaporating the solvent to dryness to obtain an oily residue;
adding an organic solvent and suspending the mixture optionally at elevated temperature; and
precipitating the crude product, collecting it by filtration, washing and drying.

7. The isolating procedure according to claim 6, wherein said evaporating the reaction mixture to dryness is carried out at a temperature of no more than 50° C.

8. The isolating procedure according to claim 6, wherein the basic aqueous solution is selected from the group consisting of aqueous solutions of sodium hydroxide, potassium hydroxide, sodium carbonate, potassium carbonate, sodium bicarbonate, and potassium bicarbonate.

9. The isolating procedure according to claim 8, wherein the said basic aqueous solution is an aqueous solution of sodium hydroxide.

10. The isolating procedure according to claim 9, wherein the concentration of the said aqueous solution of sodium hydroxide is at least 10%, preferably of about 45%.

11. The isolating procedure according to claim 6, wherein the organic solvent used for precipitating the crude product and for washing is selected from the group consisting of methyl acetate, ethyl acetate, propyl acetate, isopropyl acetate, butyl acetate, isobutyl acetate, toluene, dichloromethane, chloroform, and mixtures thereof.

12. The isolating procedure according to claim 11, wherein the organic solvent used for precipitating the crude product and for washing is ethyl acetate.

13. A procedure for converting pramipexole base to pramipexole dihydrochloride or its optical isomeric mixture as defined hereinabove to the corresponding dihydrochloride salt, the procedure comprising:
dissolving pramipexole base or its optical isomeric mixture as defined hereinabove in a solvent and filtering the solution to obtain a filtrate;
adding a solution of an inorganic acid in an organic solvent and mixing;
cooling to a reduced temperature and mixing to obtain a precipitate; and
collecting the precipitate by filtration, washing and drying.

14. The process according to claim 13, wherein the solvent for dissolving pramipexole base or its optical isomeric mixture as defined hereinabove i.e. (R,S)-2-amino-6-propyl-4,5,6,7-tetrahydrop-benzothiazole is selected from the group consisting of methanol, ethanol, 1-propanol, 2-propanol, water, and mixtures thereof.

15. The process according to claim 14, wherein the solvent is ethanol.

16. The process according to claim 13, wherein the solution of an inorganic acid in an organic solvent is a solution of at least 10% HCl in 2-propanol and preferably about 14.6% HCl in 2-propanol.

17. The process according to claim 13, wherein the solvent used for washing the precipitate is ethanol.

18. A process, according to claim 13, for purifying pramipexole dihydrochloride or the dihydrochloride salt of its optical isomeric mixture as defined hereinabove by re-crystallization from an organic solvent, the process comprising:
converting pramipexole dihydrochloride or the dihydrochloride salt of its optical isomeric mixture as defined hereinabove to the corresponding free base by treatment with at least a stoichiometric equivalent of a suitable organic or inorganic base;
converting the pure free base product again to the corresponding pramipexole dihydrochloride or the dihydrochloride salt of its optical isomeric mixture as defined hereinabove; and
isolating the purified pramipexole dihydrochloride or (R,S)-2-amino-6-propyl-4,5,6,7-tetrahydrobenzothiazole dihydrochloride.
